(19) United States
(12) Patent Application Publication
(10) Pub. No.: US 2011/0278370 A1
(43) Pub. Date: Nov. 17, 2011

(54) AROMA RELEASING PACKAGE WITH MOVEABLY ENGAGEABLE PORTIONS

(75) Inventor: James Anthony Glydon, Cedar Knolls, NJ (US)

(73) Assignee: Kraft Foods Global Brands LLC, Northfield, IL (US)

(21) Appl. No.: 13/133,425

(22) PCT Filed: Nov. 25, 2009
(86) PCT No.: PCT/US2009/065870

§ 371 Date: Jul. 28, 2011

Related U.S. Application Data
(60) Provisional application No. 61/121,606, filed on Dec. 11, 2008.

(51) Int. Cl. A61L 9/04

(52) U.S. Cl. 239/6; 239/34

(57) ABSTRACT

Packages with at least two moveably engageable portions have one or more aroma providing substances placed at one or more contact points on the portions such that the one or more aroma providing substances are released when the portions are moved relative to each other. The packages can also include one or more aroma providing substances on one or more exterior surfaces of the packages so as to provide a consumer with an immediate sensory impression.

### CROSS REFERENCE TO RELATED APPLICATIONS

This application claims priority to U.S. Provisional Patent Application No. 61/121,606 filed on Dec. 11, 2008, the contents of which is incorporated herein by reference in its entirety.

### FIELD

The present invention is directed generally to packages with moveably engageable portions that have one or more aroma providing substances applied to one or more contact points on the moveably engageable portions such that an aroma is released due to movement of the package portions relative to each other or relative to other portions of the packages. More particularly, the packages with moveably engageable portions can contain edible products such as confectionery where the released aromas can represent, identify, enhance, complement, or contrast with the anticipated flavor or functional benefit experience of the edible products.

### BACKGROUND

Aroma is generally considered to include perceptions created by the sense of smell. The pleasantness of many foods is contributed in large part to their aroma as perceived by the human sense of smell. The flavor of an edible product is made up of five tastes (sweet, salt, bitter, sour, and umami) along with the hundreds of aromas detected by the sense of smell. Smell is an especially important sense for humans as smell can have both physical and emotional effects. Additionally, humans have olfactory memories whereby certain experiences become associated with certain smells such that perceiving the smell triggers the memory. For example, smelling the aroma from a favorite food can trigger pleasant memories and trigger a desire to consume that food.

Packages with moveably engageable portions can include structures such as blister cards with either open end or closed end sleeves. Commercial examples of blister card structures include pellet chewing gums such as those that go by the brand names DENTYNE™, TRIDENT™, and others. These packages offer convenience and portability but require removal and often ingestion of the product to be able to begin perceiving the flavor experience of the product.

Aroma providing substances are known for inclusion in materials such as paper and cloth. Examples include “scratch and sniff” materials where rupturable microcapsules of aroma compounds are embedded in paper or cloth such that when the paper or cloth is scratched, the microcapsules break to release the aroma material.

It is desirable to enhance packages with moveably engageable portions with one or more aroma providing substances located at one or more contact points on the moveably engageable portions such that an aroma is released when the moveably engageable portions of the package are moved relative to each other or are moved relative to other portions of the package.

### SUMMARY

In one embodiment, there is provided a package comprising a first portion having a first contact point and a second portion moveably engageable with the first portion and having a second contact point. Additionally, a first aroma providing substance is applied to at least one of the contact points and a second aroma providing substance is applied to at least one exterior surface of the package.

In another embodiment, there is provided a package comprising a first portion having at least one first wall and having a first contact point, and a second portion moveably engageable with the first portion and having at least one second wall and having a second contact point. Additionally, a first aroma providing substance is applied to at least one of the contact points and a second aroma providing substance is applied to at least one exterior surface of the package.

In another embodiment, there is provided a method of making a package. The method comprises the steps of providing a first portion having one or more contact points; providing a second portion moveably engageable with the first portion where the second portion has one or more second contact points; applying a first aroma providing substance to at least one of the contact points; and applying a second aroma providing substance to at least one exterior surface of the package.

In another embodiment, there is provided a method of enhancing the sensory perception of a food. This method comprises the steps of providing a package comprising a first portion having a first contact point; and a second portion moveably engageable with the first portion, where the second portion has one or more second contact points; wherein a first aroma providing substance is applied to at least one of the contact points; and placing the food into the package.

In another embodiment, there is provided a method of releasing at least one aromas. The method comprises the steps of providing a package comprising a first portion having one or more first contact points; and a second portion slidably engageable with the first portion, where the second portion has one or more second contact points; wherein a first aroma providing substance is applied to at least one of the contact points; and moving the first portion so as to release the first aroma providing substance.

In another embodiment, there is provided a package comprising a first portion having one or more first contact points; and a second portion pivotably engageable with the first portion where the second portion has one or more second contact points; wherein the first portion at least partially surrounds the second portion; and wherein a first aroma providing substance is applied to at least one of the contact points.

In another embodiment there is provided a package comprising a first portion having one or more first contact points; and a second portion slidably engageable with the first portion where the second portion has one or more second contact points; wherein the first portion at least partially surrounds the second portion; and wherein a first aroma providing substance is applied to at least one of the contact points.

In another embodiment, there is provided a package comprising a first portion having one or more first contact points; and a second portion slidably engageable with the first portion where the second portion has one or more second contact points; wherein the first portion is a sleeve at least partially surrounding the second portion; and wherein the second portion is a blister card; and wherein a first aroma providing substance is applied to at least one of the contact points.

In another embodiment, there is provided a package comprising a packaging material including an aroma providing substance wherein the aroma providing substance is releasable upon tearing.

In another embodiment, there is provided a method of transferring aroma comprising the steps of providing a package comprising a first portion, the first portion having a first contact point; and a second portion, the second portion moveably engageable with the first portion, and the second portion having a second contact point; applying a first aroma providing substance to at least one of the contact points; and releasably applying a second aroma providing substance to at least one exterior surface of the package.

### BRIEF DESCRIPTION OF THE DRAWINGS

FIG. 1 shows an open-ended blister package with moveably engageable sleeve and blister card portions and one or more aroma providing substances located at one or more contact points on the sleeve and blister card portions.

FIG. 2 shows a closed-end tray package with moveably engageable sleeve and tray portions and one or more aroma providing substances located at one or more contact points on the sleeve portion.

FIG. 3 shows a sliding lid package with moveably engageable lid and tray portions and one or more aroma providing substances located at one or more contact points on the tray portion.

FIG. 4 shows an envelop package with moveably engageable flap and slot portions and one or more aroma providing substances located at one or more contact points on the flap and slot portions.

FIG. 5 shows an envelop package with alternative moveably engageable flap and slot portions and one or more aroma providing substances located at one or more contact points on the flap and slot portions.

FIG. 6. shows an open-ended blister package with moveably engageable sleeve and blister card portions and two or more aroma providing substances located at one or more contact points on the blister card portion that can be alternatively released based on the directional movement of the sleeve and blister card portions.

FIG. 7 shows a pivotable package with moveably engageable lid and tray portions and one or more aroma providing substances located at one or more contact points on the lid and tray portions.

FIG. 8 shows an open-ended blister package with moveably engageable sleeve and blister card portions and one or more aroma providing substances located at one or more contact points on blister card portions and with one or more aroma providing substances on the lidding film.

### DETAILED DESCRIPTION

In some embodiments, packages for confectionery products have multiple portions that are moveably engageable relative to each other or relative to other portions of the packages such that one or more aroma providing substances located at one or more contact points on the portions are released when the portions are moved relative to each other or relative to other portions of the packages. Release can be the result of pressure exerted on microcapsules housing the one or more aroma providing substances causing the microcapsules to rupture and release the one or more aroma providing substances. In addition to the aroma providing substances located at contact points on the package portions, in some embodiments, aroma providing substances are located on at least one exterior surface of the package.

In some embodiments, release of an aroma providing substances located at either contact points and/or exterior surface(s) offer a user of a package an early indication of what taste, flavor, or functional benefit experience she can expect from an edible product contained within the package. This early indication enhances the eating and/or functional benefit experience as the user need not wait until placing the product in the mouth to begin experiencing the product. The aroma providing substances can represent, identify, enhance, complement, or contrast with the anticipated flavor or functional benefit experience of the edible products. In embodiments where the one or more aroma providing substances are located on the package exterior surface(s), a user of the package can come into contact with the one or more aroma providing substances and thus the aroma providing substances can remain with the user after the user interacts with the package. For example, a user can handle a package of confectionery products to which aroma providing substances have been applied to at least one exterior surface and at least one contact point on moveably engageable portions of the package so as to remove individual piece(s) of confectionery from the package.

In some embodiments, the aroma providing substance has been provided in the form of at least one rupturable capsule. During package handling, the user can exert pressure on the aroma providing substance applied to at least one exterior surface in the form of at least one rupturable capsule causing the at least one rupturable capsule to break. Upon breaking, the aroma providing substance can be released such that the aroma providing substance is transferred from the package exterior to the user. For example, a user may handle the package by picking it up, moving the moveably engageable portions of the package so as to open the package and allow the user access to the product(s) housed within the package. During the handling process, the user may contact at least one exterior surface with sufficient pressure to rupture the at least one rupturable capsules thereby cause the aroma providing substance to be released at which time the aroma providing substance will come into contact with the user and thus be transferred to the user. With the aroma providing substance transferred to the user, the user can then perceive the aroma providing substance in the absence of the package.

In some embodiments, manipulating the package so as to remove individual piece(s) of confectionery can cause the at least one rupturable capsule applied to the at least one contact point to rupture thus releasing the aroma providing substance such that the user perceives the aroma providing substance.

In some embodiments, the user can receive an early indication of what taste, flavor, or functional benefit experience she can expect from the confectionery product when she opens the package causing release of the aroma providing substance and the user can continue to receive an indication about the confectionery product after she finishes using the package because the aroma providing substances located on the at least one exterior surface will be transferred to her hands, clothing, handbag, backpack, or other personal articles coming in contact with the package.

For example, a confectionery product with a spearmint flavor profile could be packaged in a package with blister card and sleeve portions where an aroma providing substance is located on one or more contact points on the blister card. In use, a spearmint aroma would be released when a user slides the blister card relative to the sleeve. The user would then begin to experience a spearmint eating experience at least several seconds before beginning to eat the product. In embodiments where spearmint aroma providing substances are also located on at least one exterior surface of the blister card or sleeve portions, the spearmint aroma providing substances would be transferred to the user when the user touches the exterior surface containing the spearmint aroma providing substance. In that way, the user of the package would also experience a spearmint eating experience after using the package as the spearmint aroma providing substance would remain on the user's hand(s), clothing, handbag, backpack, or other personal articles coming in contact with the package.

In some embodiments, release of an aroma providing substance offers a user of a package a taste or flavor experience that complements the taste or flavor of an edible product contained within the package. This complementary aroma can enhance the eating experience by supplementing the taste or flavor of the edible product.

“Complementary” aroma providing substances as used herein are those aroma providing substances that are in the same flavor family or that are in a similar flavor family. For example, complementary aroma providing substances can be in the mint flavor family or can be in the fruit flavor family. Complementary aroma providing substances can also include components that are in the same or similar sensation family, for example the cooling family, the warming family, the tingling family; or can include components that are in the same or similar taste family, for example, the sweet family, the sour family, the bitter/astringent family, the salty family, the umami family, or the kokumi family. Complementary aroma providing substances can also include components that are in the same or similar functional family, for example, the breath freshening functional benefit family or other oral care functional benefit family. The terms “family” or “type” are used interchangeably herein when referring to complementary components.

For example, a confectionery product with a blended mint flavor profile could be packaged in a package with blister card and sleeve portions where an aroma providing substance is located on one or more contact points on the blister card. In use, a menthol aroma would be released when a user slides the blister card relative to the sleeve. The initial menthol aroma would complement the blended mint flavor profile and provide the user with a minty experience at least several seconds before beginning to eat the product. In embodiments where aroma providing substances are located on at least one exterior surface of the package, a either a menthol or a blended mint aroma could be applied and would be transferred to the user's hand(s), clothing, handbag, backpack, or other personal articles coming in contact with the package during handling or other use, and thus remain with the user after handling or other use of the package. Because menthol and blended mint are in the same flavor family, the aroma providing substances would provide a complementary experience.

In another example, a confectionery product with a strawberry flavor profile could be packaged in a package with blister card and sleeve portions where an aroma providing substance is located on one or more contact points on the blister card. In use, a lime aroma would be released when a user slides the blister card relative to the sleeve. The initial lime aroma would complement the strawberry flavor as both flavors are from the fruit flavor family and provide the user with a delightfully different and yet complementary experience at least several seconds before beginning to eat the product. In embodiments where aroma providing substances are located on at least one exterior surface of the package, a either a lime or a strawberry or a complementary strawberry-lime aroma could be applied and would be transferred to the user's hand(s), clothing, handbag, backpack, or other personal articles coming in contact with the package during handling or other use, and thus remain with the user after handling or other use of the package.

In some embodiments, release of an aroma providing substance offers a user of a package a taste or flavor experience that contrasts with the taste or flavor of an edible product contained within the package. This contrasting aroma can enhance the eating experience by augmenting the taste or flavor of the edible product.

“Contrasting” aroma providing substances as used herein are those aroma providing substances that are in distinctly different families or contain components that are in distinctly different families, or that provide distinctly different intensities. For example, contrasting aroma providing substances would be those with components in the mint flavor family and those with components in the fruit family; or those with components in the spice family and those with components in the salty family; or those with components in the mild mint family and those with components in the intense mint family, and the like.

By providing a package that communicates or otherwise indicates or represents the product's taste and/or flavor, either by complementing or contrasting the product's taste and/or flavor, either before and/or after the product is used, a user's sensory perception of the product can be enhanced because the total product experience is extended to include time periods before, during, and after use of the product. As used herein the term “communicate”, is open-ended and refers to any packaging feature that indicates or represents an aspect of the product contained within the package. Product aspects can include, but are not limited to, taste aspects, flavor aspects, functional benefit aspects, emotional aspects such as confidence, control, boredom, and the like.

In some embodiments, the release of an aroma providing substance provides a user of a package with an indication of the functional benefit to be provided by the product contained within the package. For example, a confectionery product with an oral care benefit could be packaged in a package with blister card and sleeve portions where an aroma providing substance is located on one or more contact points on the blister card. In use, a signature aroma associated with the oral care functional benefit would be released when a user slides the blister card relative to the sleeve. As used herein, a “signature aroma providing substance” is an aroma providing substance that provides an aroma profile created to communicate or otherwise indicate or represent a product benefit other than the product's taste or flavor profile. The signature aroma provided by the signature aroma providing substance would provide the user with a cue that the product will provide the oral care functional benefit experience at least several seconds before beginning to eat the product and receive the oral care functional benefit. The signature aroma providing substance can be applied to contact points so as to be released when a user manipulates the package, or signature aroma providing substances can be applied to at least one exterior surface of the package so as to be transferred to the user's hand(s), clothing, handbag, backpack, or other personal articles coming in contact with the package during handling or other use and thus remain with the user after handling or other use of the package, or both.

For example, the signature aroma could be a floral aroma created to signal mouth freshening. The user would receive the floral mouth freshening cue before consuming the product and would expect the mouth freshening benefit independent of the product flavor profile which could be fruit, mint, spice, etc. When the signature aroma providing substance is located on at least one exterior surface of the package, the signature aroma would be transferred to the user's hand(s), clothing, handbag, backpack, or other personal articles coming in contact with the package when the user handles or otherwise uses the package and thus the user would be reminded of the mouth freshening benefit after handling or other use of the package. In this way, the product could have any number of flavor varieties and still communicate mouth freshening efficacy with a single, signature, floral aroma.

By providing a package that communicates or otherwise indicates, or represents the product's benefit before and/or after the product is used, a user's sensory perception of the product can be enhanced because the total product experience is extended to include time periods before, during, and after use of the product.

In some embodiments, the package can also include indicia that communicate various product aspects. As used herein the term “indicia,” is descriptive matter synonymous with “information related to,” “information representative of,” and “information indicative of” and is open-ended and can include any words, phrases, slogans, pictures, symbols, contests, marketing campaigns, textures, colors, intensities, or other characteristics or features relating to the product. In some embodiments, the product can contain features that communicate aspects of the product. Such features can include, but are not limited to, color(s), indicia on the product, shapes, etc.

As used herein, the term “release” refers to atmospheric exposure of the one or more aroma providing substances such that the one or more aroma providing substances can be or are perceived by a person of ordinary or better olfactory capability. In some embodiments, atmospheric exposure can occur due to pressure including frictional pressure causing rupture of microcapsules containing the one or more aroma providing substances. In some embodiments, atmospheric exposure can occur due to diffusion of the one or more aroma providing substances into the atmosphere. In other embodiments, atmospheric exposure can occur due to removal of covering layer that encases the aroma providing substance. In still other embodiments, atmospheric exposure can occur due to handling pressure. Combinations of mechanisms resulting in atmospheric exposure are also contemplated.

In embodiments where the one or more aroma providing substances are housed in one or more rupturable microcapsules, the microcapsules can be formed from any suitable material and can be of any suitable size and/or shape. In some embodiments, the microcapsules have particle sizes ranging from 100 nm to 1 mm.

In some embodiments, microcapsules of different sizes are used to provide release of the aroma providing substances upon repeated movement of the moveably engageable package portions. For example, a blend of larger microcapsules (200-500 microns) with smaller microcapsules (20-50 microns) can result in rupture of the larger microcapsules and release of the aroma providing substances during the initial movements of the moveably engageable package portions. Upon later subsequent movements, the additional pressure of the repeated movement can rupture the smaller microcapsules.

In some embodiments, the ratio of various sizes of microcapsules in the blend can be used to provide release of the aroma providing substances upon repeated movement of the moveably engageable package portions. For example a blend can include from about 5% to about 10% larger microcapsules (200-500 microns), from about 40% to about 50% medium sized microcapsules (75-150 microns), and from about 40% to about 55% small microcapsules (20-50 microns) where the weight percentages are w/w by weight of the microcapsule blend. Such a blend can result in rupture and release of the larger microcapsules during the first 1-10 movements of the moveably engageable package portions while the medium sized microcapsules rupture and release during the next 10-20 movements and the small microcapsules rupture and release during the final 20-50 movements.

The microcapsules can be formed from a variety of materials as described in U.S. Pat. Nos. 3,516,846 and 4,087,376 which are incorporated herein in their entirety for all purposes. Suitable materials can include, but are not limited to, polymerized urea and formaldehyde or melamine and formaldehyde, and the like. In some embodiments, materials for forming the microcapsules can be chosen for their ability to contain the one or more aroma providing substances without loss of the aroma providing substances or deterioration of the microcapsule shell. In some embodiments, materials for forming microcapsules also can be chosen for their textural properties such that the microcapsules have the strength to endure the application process without rupture and yet have the fragility to rupture when pressure from the movement of the packaging portions and/or pressure from handling the package exerts pressure on the microcapsules.

In some embodiments, the one or more aroma providing substances may include one or more encapsulated aroma chemicals. As used herein, the term “aroma providing substance” refers to any suitable aroma chemical or blend of aroma chemicals, including flavors, flavoring materials, fragrances, or combinations thereof capable of being applied to a package. Suitable aroma providing substances can include, but are not limited to, liquid aroma chemicals provided in inks, adhesives, varnishes, films, waxes, or the like. Suitable aroma providing substances can also include, but are not limited to, solid aroma chemicals provided in inks, adhesives, varnishes, films, waxes, or the like.

In some embodiments, a package with moveably engageable portions might include packaging materials such as, but not limited to, paper, paperboard, solid bleach sulfate paperboard, polyvinyl chloride, cardboard, plastic, wax, rubber, polypropylene films, polyethylene films, metallic films, foils, composites, laminates, and the like.

Additionally, the packaging materials may be treated. Treatments can include, but are not limited to, coatings such as white clay emulsion coatings or printing or other decorative treatments. The above described aroma providing substances are applied to one or more contact points on the moveably engageable portions of packages made from these packaging materials.

For example, aroma providing substances can be included in varnishes applied to packaging materials. In some embodiments, the aroma providing substance containing varnish is applied to the entire surface of the packaging material while in other embodiments, the aroma providing substance-containing varnish is applied to less than the entire surface of the packaging material. In some embodiments, the varnishes are applied to treated surfaces. In those embodiments, the intensity of the aroma providing substances can be higher than the intensity of untreated packaging materials.

In some embodiments, a package comprises movably engageable portions. As used herein, the term “movably engageable” refers to any package configuration where two or more portions move relative to each other or relative to other portions of the packages. One or more of the moveably engageable portions also might be moveable relative to one or more other portions of the package. The movement of the packaging portions can be of any type such as, but not limited to, linear movement as in slidably engageable portions or nestably engageable portions, rotary movement as in pivotably engageable or twistedly engageable portions, or combinations of types of movement. In some embodiments, the moveably engageable portions involve cohesive or adhesive forces such as tearably engageable portions, peelably engageable portions, or combinations thereof.

In some embodiments, one or more aroma providing substances are applied to one or more contact points located on the moveably engageable portions. As used herein, the term “contact point” refers to those areas on each moveably engageable portion where the portions touch each other or can make other contact with each other during use. In some embodiments, the one or more contact points represent the entire area where the moveably engageable portions touch or make other contact with each other while in other embodiments, the one or more contact points represent a fraction of the area where the moveable engageable portions touch or make contact with each other.

In some embodiments, one or more aroma providing substances are applied to at least one exterior surface of a package. As used herein, the term “exterior surface” refers to those surfaces of a package that are capable of contact with a user of the package. In some embodiments, an exterior surface can be outside surface that comes into contact with a user when the user handles the closed package. In other embodiments, an exterior surface can come into contact with a user during operation of the package. For example, a blister card and sleeve package can have exterior surfaces on the outside surfaces of the sleeve and on the blister card where the user contacts the blister card to remove a product housed within a blister card cavity.

In some embodiments, a package resembling a postal envelope can include a flap portion and slot portion that are slidably engageable. In operation, the top and bottom surfaces of the flap can touch the upper and lower edges of the slot when the flap is slid into the slot. In some embodiments, one or more aroma providing substances are applied to one or more contact points located on only a fraction of the area of the flap that touches the slot because the one or more aroma providing substances are located on one or more contact points on just the top surface of the flap. In other embodiments, the one or more aroma providing substances are applied to contact points that represent the entire area where the flap portion touches the slot portion because the one or more aroma providing substances are located on one or more contact points on the top and bottom surfaces of the flap.

In some embodiments, two or more contact points for a moveably engageable portion are adjacent one another while in other embodiments they are not adjacent. For example, a blister card package can include a sleeve portion with two side walls and a blister card portion with two side edges. The blister card and sleeve portions are slidably engageable with the sleeve portion surrounding the blister card portion. In some embodiments, the blister card's two side edges touch the interior surface of the sleeve's side walls when the blister card is slid into the sleeve. In some embodiments, one or more aroma providing substances can be applied to one or more adjacent contact points located along one side edge of the blister card. In other embodiments, the one or more aroma providing substances can be applied to one or more non-adjacent contact points located along one side edges of the blister card. In other embodiments, the one or more aroma providing substances can be applied to one or more non-adjacent contact points located on opposite edges of the blister card.

In some embodiments, a moveably engageable package has at least two portions. As used herein, the term “portion” refers to any fractional part of a package. Suitable examples of portions can include, but are not limited to, sleeves, lids, drawers, flaps, slots, blister cards, trays, and the like.

Reference will now be made to the accompanying drawings, which assist in illustrating the various pertinent features of the packaging design. Although the invention will now be described primarily in conjunction with confectionery packaging, it should be expressly understood that the invention may be applicable to other applications where aroma release due to movement of packing portions is required/desired such as cosmetic packaging, pharmaceutical packaging, cleaning product packaging, and the like. In this regard, the following description of a confectionery packaging design is presented for purposes of illustration and description. Furthermore, the description is not intended to limit the invention to the form disclosed herein. Consequently, variations and modifications commensurate with the following teachings, and skill and knowledge of the relevant art, are within the scope of the packaging design. The embodiments described herein are further intended to explain modes known of practicing the invention and to enable others skilled in the art to utilize the invention in such, or other embodiments and with various modifications required by the particular application(s) or use(s) of the packaging design.

In some embodiments, the user of a package with moveably engageable portions where one or more aroma providing substances have been applied to one or more contact points on those portions perceives an aroma when she moves the package portions relative to each other or relative to other portions of the packages. The aroma is due to release of the one or more aroma providing substances when pressure from the movement ruptures microcapsules housing the one or more aroma providing substances. The aroma can be indicative of the product contained within the package or can be any aroma designed to invoke a response to the product such as when a pleasant olfactory memory is triggered.

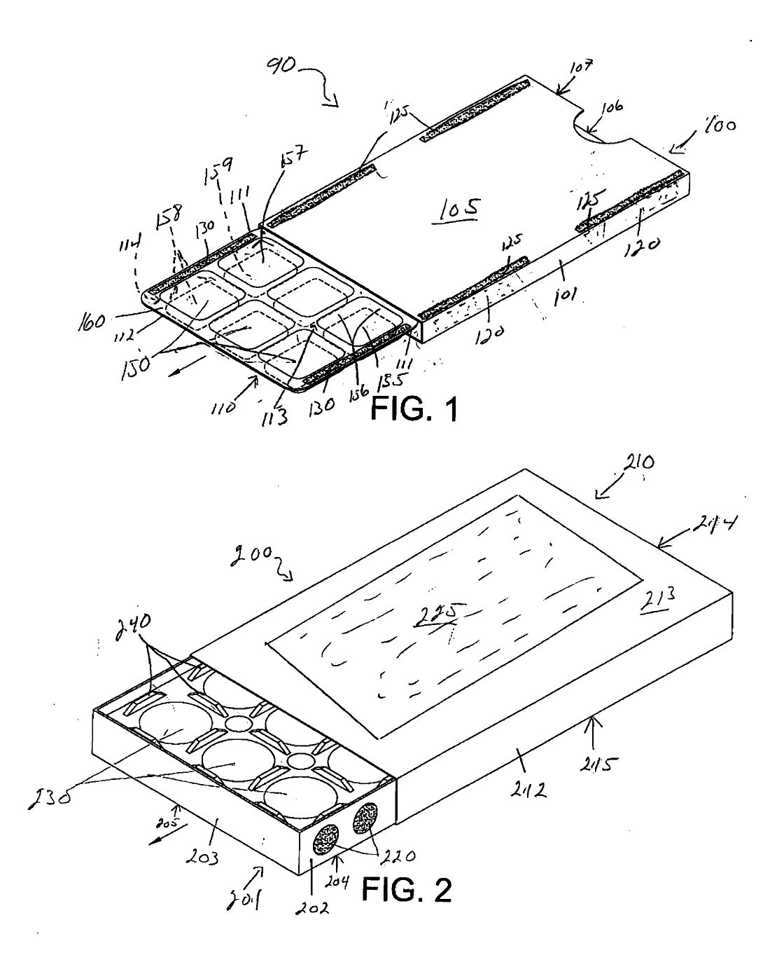

In some embodiments, the package includes at least two portions that are capable of moving relative to each other. In an embodiment as shown in FIG. 1, the package 90 can be an open-ended blister package. This open-ended blister package 90 can include a first sleeve portion 100 and a second blister card portion 110.

The first sleeve portion 100 surrounds the second blister card portion 110 and can be formed from a sheet that is scored and folded to provide two side walls: side wall 101 and a side wall opposite side wall 101 (not shown); a back wall 105; and a front wall opposite back wall 105 (not shown). The side walls, front wall, and back wall have interior, inward facing surfaces 106 and exterior, outward facing surfaces 107. The sleeve portion 100 can include a seam (not shown) where the edges of the flat sheet meet when folded. This seam can be glued or otherwise adhered to form the sleeve. The seam can fall along the side walls, front or back walls. In some embodiments, the seam falls along one side wall. Alternatively, the sleeve can be formed without a seam.

The second blister card portion 110, has four edges including side edges 111, end edge 112 and an end edge opposite to end edge 112 (not shown), a back face 113 and a front face 114. The two side edges each have a length and the two end edges each have a width. In some embodiments, the lengths are longer than the widths forming a rectangular blister card while in other embodiments, the lengths are the same as the widths forming a square blister card.

The blister card also contains one or more compartments 150 each with a top 155 and one or more side walls 156. The one or more compartment tops 155 and side walls 156 have interior, inward facing surfaces 157 and exterior, outward facing surfaces 158. The exterior facing surfaces 158 of the compartments create at least part of the front face 114 of the blister card 110. The one or more compartments 150 will have dimensions (height, length, and width) suitable for holding products such as confectionery. In some embodiments, the dimensions of the one or more compartments are sized to allow for minimal product movement. The one or more compartments are positioned a suitable distance away from the edge of the blister card such that the blister card has a rim 160 along all four edges and can have a rim between the compartments in embodiments with two or more compartments.

In some embodiments, the compartments can have indicia (not shown) on the outward facing surface of the compartment tops 159. The blister card can also include a lidding material (not shown) on the back face of the blister card that forms the bottom of the one or more compartments. The lidding material can be adhered to the back face of rim 160 to retain the product in the one or more compartments.

The sleeve and blister card portions can touch or can be in contact with each other in a number of places. In some embodiments, the width of the blister card end edge 112 and the blister card end edge opposite to end edge 112 (not shown), and the width of the sleeve back wall 105, and the sleeve front wall opposite back wall 105 (not shown) can be sized so that the side edges of the blister card 111 are in contact with the inward facing surfaces 106 of the sleeve side wall 101 and the side wall opposite side wall 101 (not shown). This creates one or more first contact points on inward facing surface of the sleeve side walls. A first aroma providing substance 120 can be applied to the one or more first contact points located on the inward facing surface 106 of sleeve side wall 101, and/or the sleeve side wall opposite sleeve side wall 101 (not shown). A second aroma providing substance 125 can be applied to at least one exterior surface of sleeve back wall 105.

In some embodiments, the height of the one or more compartments 150 can be sized so that the exterior surfaces of the one or more compartment tops 159 are in contact with the interior surface 106 of the sleeve front wall (not shown) and the back facing of the blister card 113 is in contact with the interior surface 106 of the sleeve back wall 105. This creates one or more second contact points on at least one of the exterior surfaces of the one or more compartment tops 159, the back facing of the blister card 113, the interior surface 106 of the front wall (not shown), and the interior surface 106 of the back wall 105 of the sleeve. A third aroma providing substance 130 can be applied to the one or more second contact points located on the back facing 113 of the blister card 110.

In some embodiments, a first aroma providing substance can be applied to the one or more first contact points, or a third aroma providing substance can be applied to the one or more second contact points, or a first aroma providing substance can be applied to the one or more first contact points and a third aroma providing substance can be applied to the one or more second contact points.

Aromas are then released when pressure due to the movement of the blister card portion being pulled out of or pushed in to the sleeve portion causes microcapsules housing the one or more aroma providing substances to rupture and/or when a user comes into contact with the microcapsules housing the one or more aroma providing substances located on at least one exterior surface. The blister card portion and the sleeve portion are slidably movable in two directions as the sleeve portion is open ended. Additionally, because the sleeve portion at least partially surrounds the blister card portion, the portions are slidably nestable.

The first aroma providing substance 120 can be the same as or different from the second aroma providing substance 125, which can be the same or different from the third aroma providing substance 130. The first, second, and third aroma providing substances can be complementary or contrasting.

The first and second aroma providing substances can communicate or otherwise indicate or represent a product aspect such as taste and/or flavor, either by complementing or contrasting the product's taste and/or flavor, either before and/or after the product is used. Thus, a user's sensory perception of the product can be enhanced because the total product experience is extended to include time periods before, during, and after use of the product. As mentioned above, product aspects can include, but are not limited to, taste aspects, flavor aspects, functional benefit aspects, emotional aspects such as confidence, control, boredom, and the like.

For example, a package designed for a confectionery product with a strawberry lime flavor could include application of an aroma providing substance 120 capable of releasing a strawberry aroma and application of an aroma providing substance 130 capable of releasing a lime aroma resulting in the simultaneous release of strawberry and lime aromas when the blister card portion is moved relative to the sleeve portion. In some embodiments, the package could also include an aroma providing substance 125 capable of releasing a strawberry-lime aroma.

In another blister card and open-ended sleeve embodiment as shown in FIG. 6, two or more aroma providing substances can be alternatively released based on the direction of the movement of the blister card relative to the sleeve. Similar to FIG. 1, the blister card and sleeve package 600 in FIG. 6 includes a first sleeve portion 601 and two second blister card portions: 610 and 620 adjacent to one another. In some embodiments, the two blister card portions are connected. In some embodiments, they may be connected by a perforated seam that allows a user to separate the blister card portions by tearing them apart. Blister card portion 610 can be on the left-hand side when the package 600 is viewed from the back wall 605 as in FIG. 1 while blister card portion 620 is on the right-hand side. As can be appreciated from reading the description of FIG. 1, a first aroma providing substance 630 is located at one or more first contact points on the exterior surface 611 of the back face of blister card portion 610, while second aroma providing substance 635 is located on the exterior surface of back wall 605, and third aroma providing substance 640 is located at one or more second contact points on exterior surface 621 of the back face of blister card portion 620. As in FIG. 1, the contact points are created by sizing the height of blister card compartments 650 and 660 and the height of sleeve side walls including side wall 602 and the side wall opposite side wall 602 (not shown) so that the exterior surface 611 and 621 of the back face of blister card portions 610 and 620 touches the interior surface (not shown) of sleeve back wall 605.

In this embodiment, the portions are slidably engageable and the first and third aroma providing substances are alternatively released depending on the direction of the package portion movement such that when the first aroma providing substance is different from the third aroma providing substance, a different aroma will be released depending on the direction of the movement of the package portions. As described above, pressure from the movement of the package portions and/or from when a user comes into contact with the at least one exterior surface causes microcapsules housing the one or more aroma providing substances to rupture thus releasing the aroma providing substances.

For example, aroma providing substance 630 could be capable of providing a strawberry aroma while aroma providing substance 640 could be capable of providing a lime aroma. In that case, aromas would be released alternatively depending on which direction portions 610 and 620 were moved. In some embodiments, a confectionery product with strawberry flavor can then be housed in the compartments 650 of blister card portion 610 while a confectionery product with lime flavor can be housed in the compartments 660 of blister card portion 620. In other embodiments, a strawberry lime flavored confectionery product can be housed in compartments 650 and 660. In some embodiments, aroma providing substance 635 could be capable of providing a strawberry-lime aroma.

As shown in FIG. 2, a package with two or more moveably engageable portions 200 can include a first tray portion 201 and a second closed-end sleeve portion 210. The closed-end sleeve portion 210 surrounds the tray portion 201 and can be formed from a sheet (not shown) that is scored and folded to provide two side walls: a side wall 212 and a side wall opposite side wall 212 (not shown); a front wall 213; a rear wall opposite front wall 213 (not shown); and a back wall opposite the sleeve opening (not shown). The side walls including side wall 212, the side wall opposite side wall 212 (not shown), the rear wall (not shown), front wall 213, and the back wall (not shown) have interior, inward facing surfaces (not shown). Front wall 213 has an exterior, outward facing surface 214 and side wall 212 has an exterior, outward facing surface 215. The sleeve portion can include a seam (not shown) where the edges of the flat sheet (not shown) meet. This seam can be glued or otherwise adhered to form the sleeve. The seam can fall along the side walls, front wall or back wall surfaces. In some embodiments, the seam falls along one of the side walls. Alternatively, the sleeve can be formed without a seam. The rear wall (not shown) can include flaps folded and adhered to form the closed-end of the sleeve. Alternatively, the rear wall can include flaps folded and tucked to form the closed-end of the sleeve.

The tray portion 201, has four walls including a side wall 202 and a side wall opposite side wall 202 (not shown), a front wall 203, a back wall opposite front wall 203 (not shown). Each wall has an exterior facing surface including exterior surface 204 on side wall 202 and exterior surface 205 on front wall 203. The two side walls each have a length and the front wall and back wall each have a width. In some embodiments, the lengths are longer than the widths forming a rectangular tray portion while in other embodiments, the lengths are the same as the widths forming a square tray portion.

The tray 201 also contains one or more compartments 230 each with a bottom (not shown) and at least one side wall (not shown). The one or more compartments 230 have interior, inward facing surfaces (not shown) and exterior, outward facing surfaces (not shown). The one or more compartments 230 have dimensions (height, length, and width) suitable for holding products such as confectionery. In some embodiments, the compartments can be sized to have the same shape as the product to be contained in the compartments. For example, in some embodiments, the product can be a confectionery product with a hemispherical shape and the compartment can be a hemispherical cavity sized to hold the hemispherical confectionery product. In some embodiments, the dimensions of the one or more compartments can be sized to allow for minimal product movement. The tray portion 201 can also include a lidding material (not shown) or other covering that covers all or most of the tray to retain the product or products in the one or more compartments 230.

The tray 201 can also contain separators 240 located in between compartments 230. These separators can add structural integrity to tray 201 and can help keep products housed in compartments 230 from coming out of the compartments.

As with the sleeve and blister cards in FIG. 1 and FIG. 6, the sleeve portion 210 and the tray portion 201 can touch or can be in contact with each other in a number of places. For example, the width of the tray front wall 203 and the width of the sleeve front wall 213 can be sized so that the tray side wall exterior surfaces including exterior surface 204 are in contact with the interior surfaces of the sleeve side walls (not shown) including side wall 212. This creates one or more first contact points on the exterior surfaces of tray side walls including exterior surface 204 on tray side wall 202 and one or more second contact points on the interior surface (not shown) of the sleeve side walls including sleeve side wall 212. A first aroma providing substance 220 can be applied to the one or more first contact points on the exterior surface 204 on tray side wall 202. In some embodiments, a second aroma providing substance 225 can be applied to the exterior surface of front wall 213. Additionally, or alternatively, a third aroma providing substance (not shown) can be applied to the one or more second contact points located on the interior surface (not shown) of the sleeve side walls including side wall 212. Further, a first aroma providing substance can be applied to the one or more first contact points and a third aroma providing substance can be applied to the one or more second contact points. As above in FIG. 1 and FIG. 6, the first aroma providing substance, second aroma providing substance, and the third aroma providing substance can be the same or different, complementary or contrasting.

Aromas are then released when the tray portion 201 is pulled out of or pushed in to the sleeve portion 210 and/or when a user contacts the exterior surface of front wall 213 due to pressure causing the rupture of microcapsules housing the one or more aroma providing substances. The tray portion 201 and the sleeve portion 210 are slidably movable in one direction as the sleeve portion 210 is closed ended. Additionally, because the sleeve portion 210 at least partially surrounds the tray portion 201, the portions are slidably nestable.

As with the blister card and sleeve configuration, in some embodiments, the height of the one or more tray compartments 230 and the height of the sleeve side walls including sleeve side wall 212 and sleeve rear wall (not shown) can be sized so that the exterior surfaces (not shown) of the one or more tray compartment bottoms (not shown) are in contact with the interior surface (not shown) of the sleeve back wall (not shown). This creates one or more first contact points on the exterior surfaces (not shown) of the one or more tray compartment bottoms (not shown) and one or more second contact points on the interior surface (not shown) of the sleeve back wall (not shown). Additionally, a first aroma providing substance can be applied to the one or more first contact points on the exterior surfaces (not shown) of the one or more compartment bottoms (not shown), or a second aroma providing substance can be applied to the one or more second contact points on the interior surface (not shown) of the sleeve back wall (not shown), or a first aroma providing substance can be applied to the one or more first contact points and a second aroma providing substance can be applied to the one or more second contact points.

Similarly, the tray separators 240 and sleeve side walls including sleeve side wall 212 and sleeve rear wall (not shown) can be sized such that they have heights that cause the separators 240 to touch the interior surface (not shown) of front wall 213. As above, a first aroma providing substance can be applied to one or more first contact points on the separators 240, and/or a second aroma providing substance can be applied to the exterior surface of front wall 213, and/or a third aroma can be applied to one or more second contact points on the interior surface (not shown) of front wall 213.

As above, aromas are then released when the tray portion is pulled out of or pushed in to the sleeve portion or when a user contacts with an exterior surface of the package due to pressure causing the rupture of microcapsules housing the one or more aroma providing substances. As above, the first aroma providing substance can be the same as or different from the second and/or third aroma providing substance and the first, second, and/or third aroma providing substances can be complementary or contrasting.

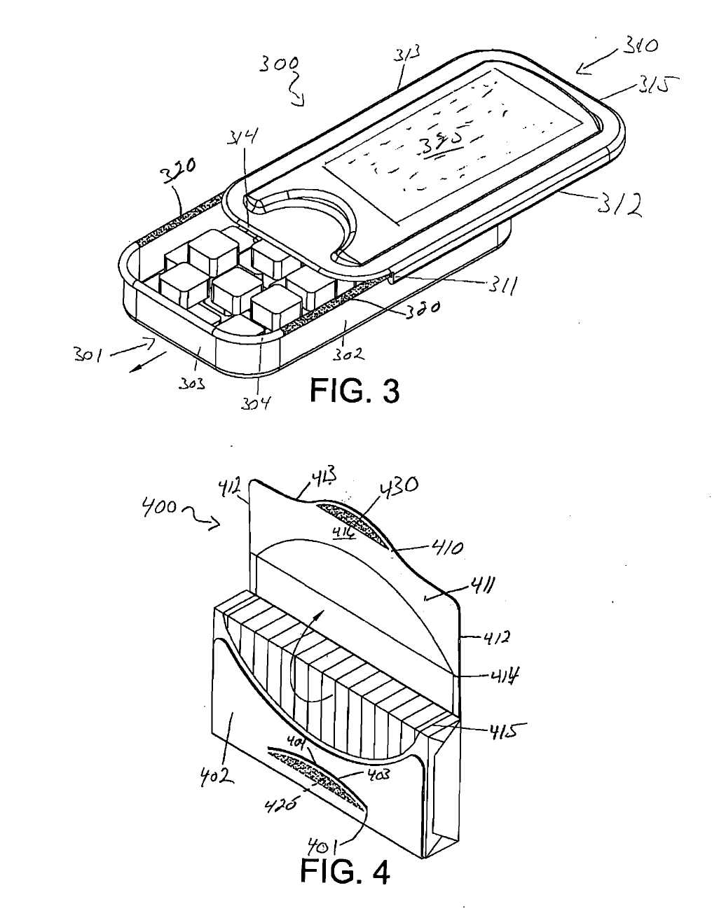

As shown in FIG. 3, a package 300 with two or more moveably engageable portions can include a first tray portion 301 and a second lid portion 310. The first tray portion 301 includes two side walls including a side wall 302 and a side wall opposite side wall 302 (not shown); a front wall 303; and a back wall opposite front wall 303 (not shown). The side walls including side wall 302 include rails 304 along the upper edge of the tray 301 sized to engage grooves 311 found on the second lid portion 310. The second lid portion 310 includes two side edges including side edge 312 and side edge 313 opposite from side edge 312; a front edge 314; and a back edge 315. The side edges 312 and 313 include groves 311 sized to engage the rails 304 on the first tray portion 301.

The rails 304 of the first tray portion 301 create one or more first contact points and the grooves 311 of the second lid portion 310 create one or more second contact points. A first aroma providing substance 320 can be applied to the one or more first contact points on the rails 304 of the tray portion 301. Additionally, or alternatively, a second aroma providing substance 325 can be applied to an exterior surface of second lid portion 310, and additionally, or alternatively, a third aroma providing substance (not shown) can be applied to the one or more second contact points located on the grooves 311 of the lid portion 310. Further, a first aroma providing substance can be applied to the one or more first contact points and a third aroma providing substance can be applied to the one or more second contact points. As above, the first aroma providing substance and the second and/or third aroma providing substance can be the same or different, complementary or contrasting.

The tray portion 301 and the lid portion 310 are linearly and slidably moveable and aroma is released when the tray and lid portions are moved relative to each other or when a user contacts the exterior surface of the lid portion 310 due to pressure causing the rupture of microcapsules housing the one or more aroma providing substances.

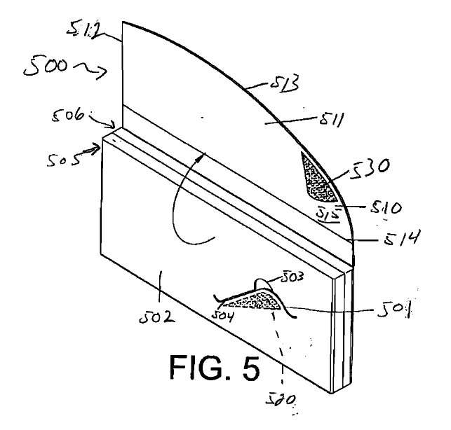

As shown in FIG. 4 and FIG. 5, packages with two or more moveably engageable portions can include packages resembling postal envelopes that include flap and slot portions. Such packages resembling postal envelopes are described in U.S. Pub. 2005/0252817 and U.S. Pub. 2006/0027483 both of which are herein incorporated by reference for all purposes. In FIG. 4, a package 400 includes a first slot portion 401 which is sized to engage a second flap portion 410. In FIG. 5, a package 500 includes a first slot portion 501 which is sized to engage a second flap portion 510.

As shown in FIG. 4, first slot portion 401 is located on a front wall 402 of a single compartment package 400 suitable for holding products such as confectionery. First slot portion 401 includes a top edge 403 and a bottom edge 404. Second flap portion 410 is located on a cover 411 that includes three adjacent side edges including two side edges 412 and a curvilinear front edge 413 running between and connecting the side edges 412 together. Cover 411 also includes a fold line 414. The cover 411 has a height from the fold line 414 to the front edge 413 that is sized to conceal the open area of the compartment defined by edge 415 when the cover 411 is folded along the fold line 414. Second flap portion 410 can be located roughly in the middle of the curvilinear front edge 413 of the cover 411 such that when the flap 410 is engaged with the slot 401, the product(s) contained in the compartment defined by edge 415 will not fall out. The second flap portion 410 includes an exterior surface (not shown) and an interior surface 416.

As shown in FIG. 5, first slot portion 501 is located on the front wall 502 of front compartment 505 which is part of a multiple compartment package suitable for holding products such as confectionery including the front compartment 505 and a back compartment 506 where front compartment 505 is connected to back compartment 506. First slot portion 501 includes a top edge 503 and a bottom edge 504. Second flap portion 510 is located on a cover 511 that includes a side edge 512 adjacent to a curvilinear front edge 513 and a fold line 514. First slot portion 501 is positioned along the front wall 502 so as to engage with the flap 510 when the cover 511 is folded along fold line 514. The second flap portion 510 is sized so that the products contained in the multiple compartments will not fall out when the flap 510 is engaged with the slot 501. The second flap portion 510 includes an exterior surface (not shown) and an interior surface 515.

The flap portions 410 or 510 and the slot portions 401 or 501 can touch or be in contact in a number of places. As in the other examples, aroma providing substances can be applied to such contact points. For example, a first aroma providing substance 420 or 520 can be applied to contact points located on the interior surface of front wall 402 or 502 below bottom slot edges 404 or 504. In use, aroma providing substances 420 or 520 can be released when pressure from the insertion of flaps 410 or 510 into slots 401 or 501 causes microcapsules housing aroma providing substances 420 or 520 to rupture. Additionally, a second aroma providing substance 430 or 530 can be applied to one or more second contact points located on the interior surface 416 or 515 of the flap portion 410 or 510. In use, aroma providing substances 430 or 530 can be released when pressure from the friction when slot bottom edges 404 or 504 engage interior surfaces 416 or 515 of flaps 410 or 510 causing microcapsules housing aroma providing substances 430 or 530 to rupture. Additionally and as in the examples above, a first aroma providing substance can be applied to the one or more first contact points and a second aroma providing substance can be applied to the one or more second contact points.

As explained above, aromas are then released when the flap 410 or 510 is inserted into or pulled out of the slot portion 401 or 501 due to pressure causing the rupture of microcapsules housing the one or more aroma providing substances. The flap portion and the slot portion are slidably movable.

The first aroma providing substance 420 or 520 can be the same as or different from the second aroma providing substance 430 or 530. The first and second aroma providing substances can be complementary or contrasting. As above, the first aroma providing substance and the second aroma providing substance can be the same or different, complementary or contrasting.

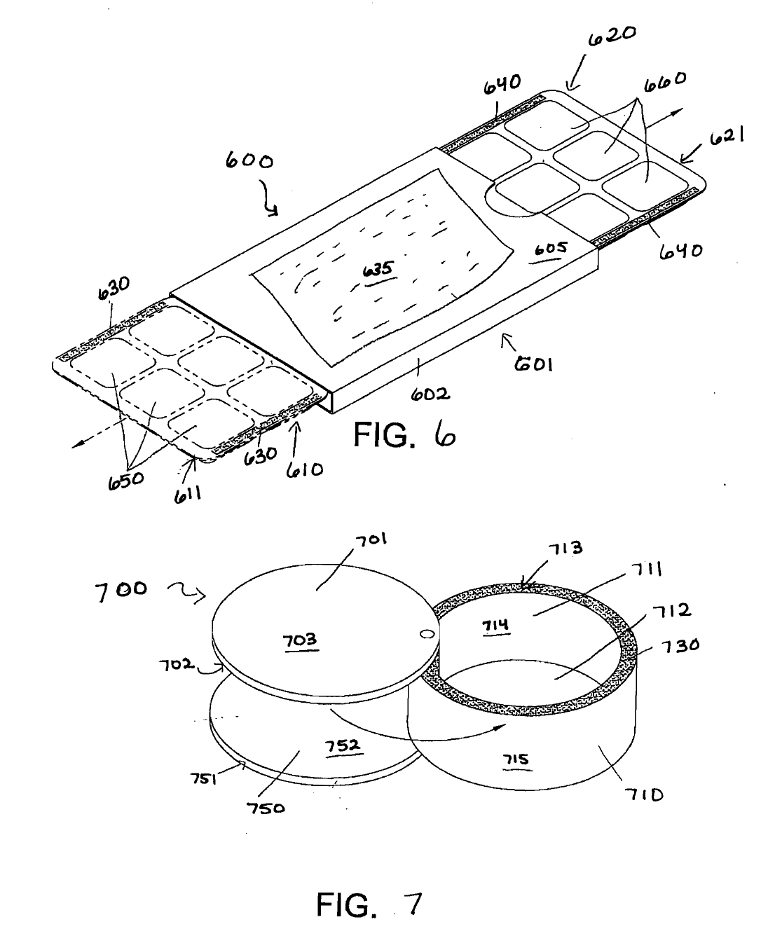

As shown in FIG. 7, package 700 includes two first lid portions: an upper lid 701 and a lower lid 750. The first lid portions 701 and 750 are pivotably moveable relative to a second container portion 710. First upper lid portion 701 includes an edge 702 that forms the periphery of upper lid portion 701 and upper lid portion 701 includes an inward facing surface (not shown) and an outward facing surface 703. Lower lid portion 750 includes an edge 751 the forms the periphery of lower lid portion 750 and lower lid portion 750 includes an inward facing surface 752 and an outward facing surface (not shown). Second container portion 710 includes at least one side wall 711, a bottom 712, and an outward facing edge 713 along the top of the at least one side wall 711. At least one side wall 711 also includes an interior surface 714 and an exterior surface 715.

The lid portions 701 and 750 and container portion 710 can touch or be in contact in a number of places. For example, the first upper lid portion 701 and/or first lower lid portion 750 can be sized to at least cover the container portion 710 when upper and/or lower lid portions 701 or 750 are in their closed positions. This creates one or more first contact points on the inward facing surface (not shown) of edge 702 of the upper lid portion 701 and one or more first contact points on the inward facing surface 752 of edge 751 of the lower lid portion 750 and one or more second contact points on the outward facing edge 713 along the top of the at least one side wall 711 of the container portion 710.

A first aroma providing substance (not shown) can be applied to the one or more first contact points on upper lid portion 701 or lower lid portion 750, or a second aroma providing substance 730 can be applied to the one or more second contact points, or a first aroma providing substance can be applied to the one or more first contact points and a second aroma providing substance can be applied to the one or more second contact points. The first and second aroma providing substances can be complementary or contrasting. As above, the first aroma providing substance and the second aroma providing substance can be the same or different, complementary or contrasting.

Aromas are then released when the upper lid portion 701 or the lower lid portion 750 is opened and/or closed due to pressure causing the rupture of microcapsules housing the one or more aroma providing substances. The lid portions 701 and 750 and the container portion 710 are pivotably movable.

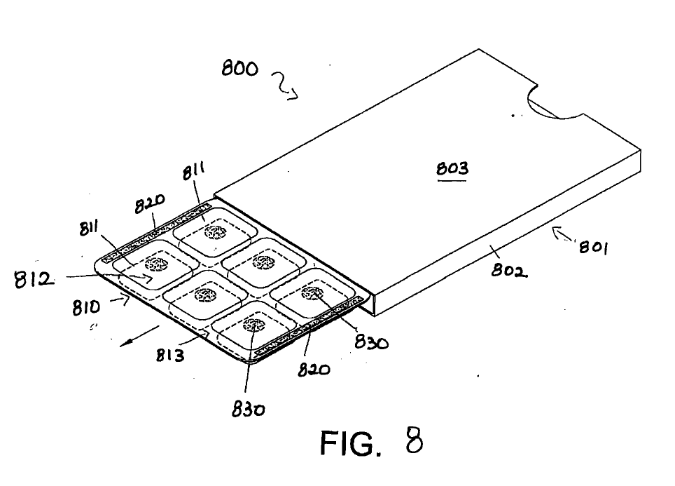

In an embodiment as demonstrated in FIG. 8 a package similar to the package shown in FIG. 1 additionally includes one or more aroma providing substances on the lidding material of the blister card portion. As can be seen from FIG. 8, package 800 can include a first sleeve portion 801 and a second blister card portion 810.

First sleeve portion 801 includes a first side wall 802, a second side wall opposite first side wall 802 (not shown), a back wall 803, and a front wall opposite back wall 803 (not shown).

Second blister card portion 810 includes compartments 811 and lidding material 812 adhered to a rim 813 that surrounds compartments 811.

As explained above in FIG. 1., contact points can be created by varying the height of the blister card compartments 811 relative to the height of the first sleeve side wall 802 and second side wall (not shown) opposite side wall 802 and/or by varying the width of blister card portion 810 relative to the width of sleeve back wall 803 and sleeve front wall (not shown) opposite sleeve back wall 803. A first aroma providing substance 820 can be placed at one or more contact points on blister tray portion 810 and a second aroma providing substance 830 can be placed on lidding material 812. This configuration can provide a sequential release of aroma as the first aroma providing substance 820 will be released initially upon movement of sleeve portion 801 relative to blister card portion 810 followed by sequential release of the second aroma providing substance 830 on lidding material 812. The sequentially released aroma providing substances can be the same or different depending on the desired sequential aroma profile.

In addition to the primary packages with moveably engageable portions as described by examples above, the packages can also have an overwrap (not shown) such as a clear film that surrounds the primary package with moveably engageable portions (not shown). The overwrap can be removed by a convenience device such as a tear strip. As described in U.S. Pat. No. 4,720,423 which is incorporated herein in its entirety for all purposes, the tear strip can be configured to release an aroma so that a consumer can receive an initial indication of what taste or flavor experience she can expect from an edible product contained within the package. This initial indication further enhances the eating experience as removal of the overwrap takes additional time when initially opening the package and the user need not wait until after additional time is spent to begin experiencing the product. Alternatively, the aroma released by the tear strip can provide an indication of freshness or can provide an aroma associated with the manufacturer or product brand or functional benefit.

### Aroma Providing Substances

In some embodiments, the aroma chemicals included in the aroma providing substance can provide an indication of the taste or flavor experience of the product. Such tastes or flavor experiences can include, but are not limited to, those flavors known to the skilled artisan, such as natural and artificial flavors. These flavorings may be chosen from synthetic flavor oils and flavoring aromatics and/or oils, oleoresins and extracts derived from plants, leaves, flowers, fruits, and so forth, and combinations thereof. Nonlimiting representative flavor oils include spearmint oil, cinnamon oil, oil of wintergreen (methyl salicylate), peppermint oil, Japanese mint oil, clove oil, bay oil, anise oil, eucalyptus oil, thyme oil, cedar leaf oil, oil of nutmeg, allspice, oil of sage, mace, oil of bitter almonds, and cassia oil. Also useful flavorings are artificial, natural and synthetic fruit flavors such as vanilla, and citrus oils including lemon, orange, lime, grapefruit, yazu, sudachi, and fruit essences including apple, pear, peach, grape, blueberry, strawberry, raspberry, cherry, plum, pineapple, apricot, banana, melon, apricot, ume, cherry, raspberry, blackberry, tropical fruit, mango, mangosteen, pomegranate, papaya and so forth. Other potential flavors include a milk flavor, a butter flavor, a cheese flavor, a cream flavor, and a yogurt flavor; a vanilla flavor; tea or coffee flavors, such as a green tea flavor, a oolong tea flavor, a tea flavor, a cocoa flavor, a chocolate flavor, and a coffee flavor; mint flavors, such as a peppermint flavor, a spearmint flavor, and a Japanese mint flavor; spicy flavors, such as an asafetida flavor, an ajowan flavor, an anise flavor, an angelica flavor, a fennel flavor, an allspice flavor, a cinnamon flavor, a camomile flavor, a mustard flavor, a cardamom flavor, a caraway flavor, a cumin flavor, a clove flavor, a pepper flavor, a coriander flavor, a sassafras flavor, a savory flavor, a Zanthoxyli Fructus flavor, a perilla flavor, a juniper berry flavor, a ginger flavor, a star anise flavor, a horseradish flavor, a thyme flavor, a tarragon flavor, a dill flavor, a capsicum flavor, a nutmeg flavor, a basil flavor, a marjoram flavor, a rosemary flavor, a bayleaf flavor, and a wasabi (Japanese horseradish) flavor; alcoholic flavors, such as a wine flavor, a whisky flavor, a brandy flavor, a rum flavor, a gin flavor, and a liqueur flavor; floral flavors; and vegetable flavors, such as an onion flavor, a garlic flavor, a cabbage flavor, a carrot flavor, a celery flavor, mushroom flavor, and a tomato flavor. These flavoring agents may be used in liquid or solid form and may be used individually or in admixture. Commonly used flavors include mints such as peppermint, menthol, spearmint, artificial vanilla, cinnamon derivatives, and various fruit flavors, whether employed individually or in admixture. Flavors may also provide the perception of breath freshening properties, particularly the mint flavors when used in combination with the cooling agents, described herein below.

In some embodiments, other flavorings include aldehydes and esters such as cinnamyl acetate, cinnamaldehyde, citral diethylacetal, dihydrocarvyl acetate, eugenyl formate, p-methylamisol, and so forth may be used. Generally any flavoring or food additive such as those described in Chemicals Used in Food Processing, publication 1274, pages 63-258, by the National Academy of Sciences, may be used. This publication is incorporated herein by reference. These may include natural as well as synthetic flavors.

Further examples of aldehyde flavorings include but are not limited to acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, .e., melonal (melon), 2,6-dimethyloctanal (green fruit), and 2-dodecenal (citrus, mandarin), cherry, grape, blueberry, blackberry, strawberry shortcake, and mixtures thereof.

In some embodiments, flavoring agents are used at levels that provide a perceptible sensory experience. In other embodiments, flavoring agents are used at levels below their threshold levels such that they do not provide an independent perceptible sensory experience. At subthreshold levels, the flavoring agents may provide an ancillary benefit such as flavor and/or aroma enhancement or potentiation.

In some embodiments, a flavoring agent may be employed in either liquid form and/or dried form. When employed in the latter form, suitable drying means such as spray drying the liquid may be used. Alternatively, the flavoring agent may be absorbed onto water soluble materials, such as cellulose, starch, sugar, maltodextrin, gum arabic and so forth or may be encapsulated. In still other embodiments, the flavoring agent may be adsorbed onto silicas, zeolites, and the like.

In some embodiments, the flavoring agents may be used in many distinct physical forms. Without being limited thereto, such physical forms include free forms, such as spray dried, powdered, beaded forms, encapsulated forms, and mixtures thereof.

### Aroma Providing Substance Application Methods

In some embodiments, the aroma providing substance is applied to contact points located on the moveably engageable packaging portions using any suitable application process. Suitable application processes can include any method known to those of ordinary skill in the art. In some embodiments, the application process can include contact and non-contact printing processes. In some embodiments, printing processes such as ink jet printing, screen printing, flexographic printing, rotary screen printing, variable screen printing, silk screen printing, off-set printing, roto-gravure printing, spray printing, immersion printing, off-set lithographic printing, and the like or combinations of such printing processes may be used.

In addition to printing processes, in some embodiments, the aroma providing substance can be applied to contact points on portions of moveably engageable packages by applying a substrate including the aroma providing substance to the package portion(s). Suitable substrates can include, but are not limited to, paper and/or plastic substrates. In some embodiments, substrates can include polymers such as polypropylene or polyethylene into which aroma providing substances have been included. In other embodiments, substrates can include waxes. In still other embodiments, the aroma providing substance can be included in the substrate in an encapsulated form such as in a rupturable microcapsule.

1. A package comprising:
a first portion, said first portion having a first contact point; and
a second portion, said second portion moveably engageable with said first portion, and said second portion having a second contact point;
wherein a first aroma providing substance is applied to at least one of said contact points; and
wherein a second aroma providing substance is applied to at least one exterior surface of said package.

2.-8. (canceled)

9. The package as in claim 1, said package including a third aroma providing substance applied to at least one of said contact points.

10. (canceled)

11. The package as in claim 9, wherein said first aroma providing substance is the same as said third aroma providing substance.

12. (canceled)

13. The package as in claim 9, wherein said first aroma providing substance is not the same as said third aroma providing substance.

14.-19. (canceled)

20. The package as in claim 1, wherein said first portion is a sleeve.

21. The package as in claim 20, wherein said second portion is a blister card slidably engageable with said sleeve.

22. (canceled)

23. The package as in claim 20, wherein said second portion is a tray slidably engageable with said sleeve.

24. The package as in claim 23, wherein said first portion is closed at one end.

25. The package as in claim 1, wherein said first portion is a flap.

26. The package as in claim 25, wherein said second portion is a receiving slot slidably engageable with said flap.

27. The package as in claim 1, wherein said first portion is a lid.

28. The package as in claim 27, wherein said second portion includes at least one rail slidably enagageable with said lid.

29. The package as in claim 1, wherein said first portion and said second portion are pivotably engageable.

30. The package as in claim 1, wherein said first portion and said second portion are slidably engageable.

31. (canceled)

32. The package as in claim 1, wherein said first portion and said second portion are peelably engageable.

33.-38. (canceled)

39. The package as in claim 1, wherein said first aroma providing substance includes at least one rupturable aroma-containing capsule.

40.-41. (canceled)

42. The package as in claim 1, wherein said first aroma providing substance is releasable upon movement of said first portion relative to said second portion.

43. The package as in claim 42, wherein said release occurs more than once.

44.-74. (canceled)

75. A method of enhancing the sensory perception of a food comprising the steps of:
providing a package comprising:
i. a first portion, said first portion having a first contact point; and
ii. a second portion, said second portion moveably engageable with said first portion, and said second portion having a second contact point;
iii. wherein a first aroma providing substance is applied to at least one of said contact points; and
placing said food into said package.

76.-79. (canceled)

80. A method of releasing aroma comprising the steps of:
providing a package comprising:
i. a first portion, said first portion having a first contact point; and
ii. a second portion, said second portion slidably engageable with said first portion, and said second portion having a second contact point;
iii. wherein a first aroma providing substance is applied to at least one of said contact points; and
moving said first portion so as to release said aroma providing substance.

81.-102. (canceled)
